(19) United States
(12) Patent Application Publication
(10) Pub. No.: US 2014/0066494 A1
(43) Pub. Date: Mar. 6, 2014

(54) ACNE TREATMENT

(71) Applicant: Christos C. Zouboulis, Berlin (DE)

(72) Inventor: Christos C. Zouboulis, Berlin (DE)

(21) Appl. No.: 13/717,011

(22) Filed: Dec. 17, 2012

Related U.S. Application Data
(63) Continuation of application No. 13/459,725, filed on Apr. 30, 2012.
Application No. 13/459,725 is a continuation of application No. 12/500,558, filed on Jul. 9, 2009, now abandoned.
Application No. 12/500,558 is a continuation of application No. 10/476,405, filed on Aug. 2, 2004, now abandoned.

(30) Foreign Application Priority Data
Apr. 30, 2001 (DE) 10121252.6

(51) Int. Cl. A61K 31/381; A61K 45/06; A61K 31/19

(52) U.S. Cl. CPC A61K 31/381; A61K 31/19; A61K 45/06
USPC 514/443; 514/557

(57) ABSTRACT

The invention relates to the use of lipooxigenase inhibitors for treating acne, particularly inflammatory acne. The inventive lipooxigenase inhibitor can be used alone or in combination with other lipooxigenase inhibitors or anti-acne active agents in a pharmaceutically suitable composition, particularly through oral and/or local-topic application.

This application is a continuation of U.S. patent application Ser. No. 13/459,725, filed Apr. 30, 2012, entitled “Acne Treatment”, which is a continuation of U.S. patent application Ser. No. 12/500,558, filed Jul. 9, 2009, entitled “Acne Treatment”, which is a continuation of U.S. patent application Ser. No. 10/476,405, filed Aug. 2, 2004, entitled “Acne Treatment”, which is a 371 national phase filing of PCT/EP02/04715, filed Apr. 29, 2002, which claims priority to DE 10121252.6, filed Apr. 30, 2001. Each of the prior applications is incorporated by reference herein in its entirety.

The present invention relates to the use of active substances for the treatment of acne, especially of inflammatory acne. The active ingredients can be administered in a pharmaceutical composition in accordance with any conventional way of application.

It is assumed that acne lesions occur due to the interaction of interrelated and complex processes: an increased excretion of the sebum (Cunliffe W J, Shuster S: Pathogenesis of acne. Lancet. 1969; 1 (7597): 685-687), a ductal cornification (Holmes R L, Williams M, Cunliffe W J: Pilo-sebaceous duct obstruction and acne. Br J. Dermatol. 1972; 87: 327-332 and 35), an abnormal number and function of P. acnes (Cunliffe W J, Clayden A D, Gould D, Simpson N B: Acne vulgaris-its aetiology and treatment. A review. Clin Exp Dermatol. 1981; 6: 461-469) as well as the production of inflammatory mediators that result in the formation of papules, pustules and temporarily deep inflammatory lesions.

The initiators and real causes, respectively, of the inflammatory response in the case of acne are unclear, however, until this day. Plural factors have been taken into consideration as mediators of the inflammation. This is especially true for microorganisms, above all P. acnes (Cunliffe W J: Acne. London, Dunitz, 1989), as well as its products (Hellgren L, Selstam G, Vincent J: Prostaglandin-like substances in Propionibacterium acnes.II. Stimulatory effect on ovarian cyclic AMP. Experientia. 1979; 35: 196-197 and Webster G F, Leyden J J: Characterisation of serum independent polymorphonuclear leucocyte chemotactic factors produced by Propionibacteria acnes. Inflammation 1980; 4: 261-269), such as free fatty acids (which can be produced by a triglyceride metabolism initiated by means of follicular bacteria) and oxidative products of squalene. Accordingly, conventional concepts of therapy against acne are primarily based on topical or systemic antibiotics which result in a reduction of the follicle number of bacteria (Hubbell C G, Hobbs E R, Rist T, White J W: Efficacy of minocycline compared with tetra-cycline in treatment of acne vulgaris. Arch Dermatol. 1982; 118: 989-992; Juhlin L, Liden S: A quantitative evaluation of the effect of oxytetracycline and doxycycline in acne vulgaris. Br J. Dermatol. 1969; 81: 154-158 and Leyden J J, McGinley K J, Kligman A M: Tetracycline and minocycline treatment. Arch Dermatol. 1982; 118: 19-22). Moreover anti-inflammatory drugs have rarely been used so far for the topical or systemic treatment of different types of acne, such as corticosteroids, colchicine, resorcinol, isoniazid, topical UV light and PUVA (Cunliffe W J: Acne. London, Dunitz, 1989).

Quite a long time ago benoxaprofen was described as an anti-inflammatory drug for treating the nodular acne by way of oral administration (Cunliffe W J: Acne. London, Dunitz, 1989). In clinical tests, however, disadvantageous effects including a photosensitivity, exzema, milii, onycholysis and a hepatotoxicity with jaundice have been observed, especially in the case of older patients having an impaired renal function, so that the drug was finally withdrawn from the market (Halsey J P, Cardoe N: Benoxaprofen: Side effect profile in 300 patients. Br Med J. 1982; 284: 1365-1368 and Hindson T C, Daymond T, Diffey B, Lawlor F: Side effects of benoxaprofen. Br Med J Clin Res Ed. 1982; 284: 1368-1369).

The documents mentioned hereinafter describe particular substances and/or compounds having a number of active mechanisms, inter alia a 5-lipoxygenase inhibition, wherein, apart from a plurality of possible treatments, also acne is briefly mentioned:

WO-A-200 105 780 (Chroman-Analoga), WO-A-0 071 540 (hydrochloride of 5-(4-(6-methoxy-1-methyl-1H-benzmidazole-2-yl-methoxy)benzyl)thiazolid-in-2,4-dion for the treatment of diabetes), WO-A-0 061 582 (condensed imidazole compounds), WO-A-0 061 581 (benzimidazole or imidazole pyridine derivatives), WO-A-0 059 889 (use of α-substituted derivatives), WO-A-9 937-314 (lipid-containing extract from the sea cucumber), WO-A-9 918 081 (phenyl(oxy or thio)alkyl-substituted benzo- or pyrido-condensed imidazole compounds), U.S. Pat. No. 5,196,431 (2-substituted amino-4,6-di-tert-butyl-5-hydroxy-pyridin derivatives), U.S. Pat. No. 5,187,175 (acyl- or hydroxyl-imino-substituted hydroxypyrimidine derivatives), U.S. Pat. No. 5,142,095 (diaryl-alkanoids of particular structure), WO-A-92 13 844 (2-substituted 4,6-di-tert-butyl-5-hydroxy-1,3-pyrimidine compounds) and EP-A-449 216 (2-substituted azolidinone derivatives). However, a direct connection between a specific lipoxygenase inhibitor and an actual therapeutic effectiveness against acne, especially with human beings, is not mentioned in the a.m. documents.

Thus it was the object of the invention to extend the possibilities of therapy against acne, especially against inflammatory acne, and in so doing to possibly improve the effect while simultaneously reducing side-effects.

In accordance with the invention, the object is achieved by using at least one substance selected from the group consisting of lipoxygenase inhibitors for treating acne. In a further aspect of the invention, the at least one lipoxygenase inhibitor is used in a suited pharmaceutical composition including usual pharmaceutically acceptable carriers and further auxiliary agents, where appropriate.

Within the scope of the present invention it was surprisingly found that, contrary to the prevailing opinion of the role of P. acnes or other microorganisms as principal factor causing acne, obviously intrinsic mechanisms of human sebocytes may exert a significant influence on the formation of acne, especially of inflammatory acne. It was further found that leukotrienes of the B₄ (LTB₄) type representing 5-lipoxygenase metabolites of arachidonic acid are strong influencing factors which can probably be traced back to the role thereof as ligand of the peroxysomen-proliferator-activated receptors (PPARs) that take part in the differentiation of the sebaceous gland cells. It can be further assumed that the reduction of the expression and protein formation of pro-inflammatory cytokines, especially of cytokine IL 1α is important. There can occur the IL-8 formation by the sebaceous gland cells and thus a hemotactic movement of inflammatory cells to the sebaceous gland. Hence it was found within the scope of the invention that lipogenase inhibitors, especially those of the 5-type, can be used as excellent active ingredients for treating acne, especially the inflammatory acne.

So far, leukotrienes which have a family of lipid mediators having a plurality of pharmacological effects on the respiratory, the cardiovascular, the gastrointestinal and the cutaneous system have been taken into consideration as target of other pathophysiologic processes in animal models and with human beings, wherein conditions such as asthma, adult RDS (Respiratory Distress Syndrome), chronic bronchitis, septic shock, inflammatory intestinal disease, cancer, dermatitis, systemic lupus erythematode and psoriasis were in the foreground (cf. Lotti T M, Menchini G, Spallanzani A et al. Arachidonate transforming and immunomodulating agents: unapproved uses or indications. Clin Dermatol 2000; 18:118-123; Zhu Y I, Stller M J: Preview of potential therapeutic applications of leukotriene B4 inhibitors in Dermatology. Skin Pharmacol Appl Skin Physiol 2000; 13:235-245).

According to the invention, 5-, 8-, 12- and 15-lipoxygenase inhibitors can be used wherein the 5-lipoxygenase inhibitors are preferred.

With respect to known lipoxygenase inhibitors suited according to the invention but described for other conditions and not especially in connection with acne, in particular those of the 5-type, reference is made to the scientific and patent literature the disclosures of which are included here by way of reference.

Especially suited examples of the known 5-lipoxygenase inhibitors usable according to the invention include:

Masoprocol, Tenidap, (±)-1-(1-benzo[b]thien-2-ylethyl)-1-hydroxy-urea (Zileuton, Abbottt A-64077) Abbott A-76745, N′-[[5-(4-fluorophenoxy)f-uran-2yl]-1-methyl-2-propynyl]-N′-hydroxyurea (Abbott A-78773), (R)(+)N′-[[5-(4-fluorophenoxy)furan-2-yl]-1-methyl-2-propynyl]-N-hydroxyu-rea (Abbott A-79175), Abbott ABT 761, Dainippon AL-3264, Bayer Bay-x-1005, Biofor BF-389, bunaprolast, Ciba-Geigy CGS-25997, Cytomed CMI-392, Cytomed CMI-568, Atlantic Pharmaceutical CT3, Takeda CV-6504, Efamol EF-40, Enazadrem-phosphate, Leo Denmark ETH-615, Flezelastin-hydrochlorid-e, Flobufen, Merck Frosst L 663536, Merckle ML3000, Linazolast, Lonapalen, Mercian MER W8020, N-hydroxy-N[1-(2-phenyl-5-benzofuranyl)ethyl]urea (R.W. Johnson Research Institute), Ontazolast, 3M Pharmaceuticals R-840, Rilopirox, Hoechst Marion Roussel RU54808, Schering Plough SCH 40120, Tepoxalin, Tanabe 757, Tanabe 799, Linetastine (Terumo, TMK-688), Glaxo Wellcome WILD20, Zeneca ZD-2138, Abbott A-121798, Abbott A 72694, Abbott A-80263, Biofor BF-397, Bristol-Myers Squibb BU-4601A, Carbazoycin C, Lagunamycin, Wellcome BW-70C, Ciba-Geigy CGS-26529, Warner-Lambert CI 1004, Warner-Lambert PD-136005, Warner-Lambert PD-145246, Eisai E 3040, Fujirebio F-1322, Fisons FPL-64170, Fujisawa FR 110302, Nippon Hypox HX 0386, Merck & Co L-699333, Merck Frosst L 739010, Lilly LY269415, Lilly NY 178002, Meiji Milk MM-7002, Hoechst Roussel P 8892, Hoechst Roussel P 8977, Hoechst Roussel HP977, SmithKline Beecham SB-202235, Green Cross SS-81-OH, Terumo Keio University TMK 685, American Home Products WAY-121520, American Home Products WAY-125007, Zeneca ZD 7717, Zeneca ZM 216800, Zeneca ZM 230487, 1,2-dihydro-n-(2-thiazolyl)-1-oxopyrrolo(3,2,1-kl)phenol-thiazin-1-carboxamide, Abbott A-65260, Abbott A-69412, Abbott-63162, American Home Products AHR-5333, Bayer Bay-q-1531, Boehringer Ingelheim BI-L-357, Boehringer Ingelheim BI-L-93BS, Boehringer Ingelheim BI-L 226XX, Bristol-Myers Squibb BMY-30094, Carbazomycin B, Wellcome BW 4C, Wellcome BW-B218C, Wellcome BW-B70C, Chauvin CBS-1114, Ciba-Geigy CGS-21595, Ciba-Geigy CGS-22745, Ciba-Geigy CGS-23885, Ciba-Geigy CGS 24891, Ciba-Geigy CGS-8515, Chiesi CHF-1909, Warner-Lambert CI-986, Warner-Lambert CI 987, Cirsiliol, Docebenon, DuPont Merck DuP-654, Eisai E 5110, Eisai E-6080, Green Cross EN-105, Enofelast, Epocarbazolin-A, Eprovafen, Evandamin, Forsythiasid, Fisons FPL 62064, Glaxo GR-80907, Zeneca ICI-211965, Isoflavane, Kyowa Hakko KF-8940, Merck & Co L-651392, Merck & Co L651896, Merck & Co L-652343, Merck & Co L-656224, Merck & Co L-670630, Merck & Co L-674636, Merck & Co L-691816, Lilly LY233569, Lilly LY-280810, Merck & Co MK-591, Merck & Co MK886, Nitrosoxacin-A, Ono ONO-5349, Ono ONO-LP-219, Ono ONOLP-269, Warner-Lambert PD-127443, Purdue Frederick PF-5901, Sandoz QA-208-199, Johnson & Johnson R-68151, Johnson & Johnson R-85355, Rhone-Poulenc Rorer Rev-5367, Revlon 5901, Rhone-Poulenc Rorer RG-5901-A, Rhone-Poulenc Rorer RG-6866, Roussel-Uclaf RU-46057, Searle SC-41661A, Searle SC-45662, Sandoz SDZ-210610, SmithKline Beecham SK&F-104351, SmithKline Beecham SK&F-104493, SmithKline Beecham SK&F-105809, Synthelabo SL-810433, Teijin TEI-8005, Terumo TMK-777, Terumo TMK-781, Terumo TMK-789, Terumo TMK-919, Terumo TMK-992, Teikoku Hormone TZI-2721, Teikoku Hormone TZI-41127, American Home Products WAY-120739, American Home Products WY 47288, American Home Products WY-48252, American Home Products WY-50295, Yoshitomi Y-19432, dihydroarachidonic acid, Merck MK571, Merck MK679, IC1207,968 and IC1204,219 (ICI), SC-41930, SC-51146, SC-37920, SC-53228, SC-50605 and SC-51146 (Searle), Wako AA-681, Wellcome BW755C, KCl 1404 [(4-methyl-2-pyridinyl)-1-piperazinyl)ethyl)-4H-pyrrolo(3,2,1-ij)quinolin-e, 15-HETE, Leflunomide (HWA486), 4-acylaminophenyl derivatives, Chamazulen (chamomile extract), poly-unsaturated fatty acids, VLM295 or LY293111 (Vanguard), 4,5-dihydro-1H-1,2,4-triazolquinone adducts of the type 1a, b, c (N-adducts) and of the type 2 (C-adducts) as products of the reaction of quinones with N-alkyl- and N-arylhydrazones, terpenes with acetyl-11-keto-beta-boswellic acid (AKBA) as chemical lead (nonredox inhibitors) as well as frankincense extract.

With respect to a better effectiveness, among these 5-lipoxygenase inhibitors those are preferred which are of the non-steroidal type. Especially preferred is the 5-lipoxygenase inhibitor (+)-1-(1.benzo[b]thien-2-ylethyl)-1-hydroxyurea (Zileuton; Abbott A-64077; Zyflo®), because a minimum toxicity and a good tolerance are connected therewith.

There is furthermore preferred the extract of the Indian frankincense resin (Boswellia serrata) conventionally merely used for treating asthma which contains as a 5-lipoxygenase inhibitor with approx. 5-8% boswellic acid. Respective tablets, H15, are available in Germany from Wira Company, Goettingen.

Examples of 12-lipoxygenase inhibitors are 2-substituted anthracenone, caffeine acid amide derivatives (cf. JP-A-6247850), luteolin and chrysoerol (wherein the two latter substances at the same time also have a 5-lipoxygenase inhibitor effect).

The lipoxygenase inhibitors can be used solely or in combination of two or more inhibitors of the same or a different lipoxygenase type. What is also favorable is a combination of at least one 5-lipoxygenase inhibitor and at least one different conventional anti-acne active ingredient or drug, preferably in combination with antibiotics (local or systemic) active against acne, azelaic acid (local), benzoyl peroxide (local) and especially in combination with a retinoid compound (local or systemic).

The active ingredient can be administered through usual ways of application, e.g. in a systemic way, enteric, especially oral or rectal, transdermal, nasal by way of inhalation and parenteral, especially by injection (subcutaneous, intramuscular or intravenous) or the like. Human beings and animals such as mammals and rodents can be treated. In order to keep the systemic impairment due to the use of the active ingredient as low as possible or exclude the same, respectively, the local and especially the topical application is preferred. To this end, carrier-free systems or systems based on carriers such as plaster, dressing material etc. can be used. A particularly preferred combination therapy consists in applying 5-lipoxygenase inhibitors together with retinoids, suitably either locally in common or the 5-lipoxygenase inhibitor systemically and the retinoid compound locally. It is one advantage of this combination that retinoids act on the comedones which are not influenced by the 5-lipoxygenase inhibitors and thus both inflammatory and non-inflammatory lesions can be improved by the combination.

The lipoxygenase inhibitors can be used in pharmaceutical compositions comprising such a quantity of the lipoxygenase inhibitor that it is effective against acne, possibly as described in combination with other anti-acne-agents, in combination and admixture, respectively, with pharmaceutically acceptable drug carriers usual for the respective above-described types of application. There are suited, for instance, tablets or capsules including the active component(s) together with extenders such as, e.g., lactose, dextrose, sucrose, manitol, sorbitol, cellulose, and/or glycin, lubricating agents such as, e.g., silicon dioxide, sebum, stearic acid as well as the magnesium or calcium salts thereof and/or polyethylene glycol, for tablets furthermore binders such as, e.g., magnesium aluminum silicate, starch, gelatin, tragacanth, methyl cellulose, sodium carboxymethyl cellulose and/or polyvinyl pyrrolidone, as well as, if desired, disintegrating agents such as, e.g., starch and modified starch, agar, alginic acid and the sodium salt thereof, or mixtures thereof, and, where appropriate, absorbents, dyes, taste substances and sweeteners. Injectable compositions are, for instance, aqueous isotonic solutions or suspensions, and suppositories are preferably made of fat emulsions or suspensions. Local and/or topical pharmaceutical compositions are, for instance, ointments, creams, gels, oils, emulsions, lotions, pastes or solutions and contain, apart from the active ingredients, the additives adequate for the a.m. formulations. In the case of locally topical applications moreover agents to increase the percutaneous resorption can be added, for instance hyaluronidate, dimethyl sulfoxide (DMSO) and the like. Said pharmaceutical compositions can be sterilized and/or contain further auxiliary agents, such as preservatives, stabilizers, wetting agents, emulsifiers etc. The compositions can be prepared by means of conventional methods for mixing, granulating or coating. The active ingredient(s) may be contained in the compositions in a quantity of 0.1 to 50% by weight, preferably 1 to 40, furthermore preferred up to 20% by weight and especially up to 10% by weight, based on the total weight of the pharmaceutical compositions.

Hereinafter the present invention will be described in detail by way of an example.

### EXAMPLE

For treating the inflammatory acne 10 patients having acne papulopustolosa (m:f 6:4, age 19±5 years) were treated orally with a selective 5-lipoxygenase inhibitor, namely with (±)-1-1.benzo[b]thien-2-ylethyl)-1-hydroxyurea (Zileuton) 4×600 mg/d p.o. for a period of three months. The number of lesions and the general degree of severity index according to Allen and Schmidt (Allen B S, Smith J G: Various parameters for grading acne. Arch Dermatol. 1982; 118: 23-25) were clinically judged. Moreover, the surface lipids using Sebumeter® and the liver enzymes in the serum were determined at the beginning of the study, in the 2^{nd}, 4^{th}, 8^{th} and 12^{th} weeks of treatment and 2 weeks after completion of the therapy. The LTB4 detection in the blood by radio-immuno-assay and the lipid fractions in the serum by chromatography were examined at the beginning and in the 12^{th} week of treatment. The patients were photographed at the beginning and at the end of the treatment. The degree of severity index of acne decreased continuously and dependent on the time (41±28% of the initial value in the 12^{th} week of treatment; p<0.05). This effect occurred due to the decrease of the number of inflammatory lesions up to 29±24% (p<0.01), while comedones did not respond. Neither subjective nor objective side-effects were noticed. The total lipids in the sebum decreased significantly (35±51%, p<0.05), the pro-inflammatory free fatty acids (22±18%) and the lipoperoxides (26±30%) decreased, too. The degree of clinical improvement strongly correlated with the reduction of the total lipids in the sebum (p=0.0009, r²=0.81) and the free fatty acid (p=0.0003, r²=0.82). In contrast to that, LTB₄ in the blood and the surface lipids were not influenced. All examined parameters remained practically unchanged during the two-weeks' follow-up phase. To sum up, thus first indirect proofs could be furnished for a genuine inflammatory etiology of acne. Moreover the systemic inhibition of the arachidonic acid metabolism resulted in the decrease of the total lipids and the pro-inflammatory lipid fractions in the sebum which are considered to be responsible for the development of the inflammatory acne lesions (Brom J, Konig W. Cytokine-induced (interleukins-3, -6 and -8 and tumor necrosis factor-beta) activation and deactivation of human neutrophils: Immunology. 1992; 75: 281-285 and Doran T I, BaffR, Jacobs P, Pacia E: Characterization of human sebaceous cells in vitro. J Invest Dermatol. 1991; 96: 341-348).

Consequently, it is possible according to the invention by the use of lipoxygenase inhibitors, especially those of the 5-type, to effectively bring about a significant improvement of the acne symptoms while non-inflammatory lesions are not impaired. At the same time, a reduction of sebaceous gland lipids is achieved while at the same time pro-inflammatory sebum lipids are inhibited. On the other hand, the missing influence of the LTB₄ level in the blood has an advantageous effect, because it points at missing systemic effects. In fact, with all treated patients no negative reactions were observed.

1. A method of treating acne comprising administering an effective amount of at least one lipoxygenase inhibitor to a mammal in need thereof.

2. The method of claim 1, wherein the lipoxygenase inhibitor is a 5-lipoxygenase inhibitor.

3. The method of claim 2, wherein the 5-lipoxygenase inhibitor is selected from the group consisting of: Masoprocol, Tenidap, (±)-1-(1-benzo[b]thien-2-ylethyl)-1-hydroxyurea (Zileuton, Abbott A-64077) Abbott A-76745, N′-[[5-(4-fluorophenoxy)furan-2yl]-1-methyl-2-propynyl]-N′-hydroxyurea (Abbott A-78773), (R)(+)N′-[[5-(4-fluorophenoxy)furan-2-yl]-1-methyl-2-propynyl]-N-hydroxyu-rea (Abbott A-79175), Abbott ABT 761, Dainippon AL-3264, Bayer Bay-x-1005, Biofor BF-389, bunaprolast, Ciba-Geigy CGS-25997, Cytomed CMI-392, Cytomed CMI-568, Atlantic Pharmaceutical CT3, Takeda CV-6504, Efamol EF-40, Enazadrem-phosphate, Leo Denmark ETH-615, Flezelastin-hydrochlorid-e, Flobufen, Merck Frosst L 663536, Merckle ML3000, Linazolast, Lonapalen, Mercian MER W8020, N-hydroxy-N[1-(2-phenyl-5-benzofuranyl)ethyl]urea (R.W. Johnson Research Institute), Ontazolast, 3M Pharmaceuticals R-840, Rilopirox, Hoechst Marion Roussel RU54808, Schering Plough SCH 40120, Tepoxalin, Tanabe 757, Tanabe 799, Linetastine (Terumo, TMK-688), Glaxo Wellcome WILD20, Zeneca ZD-2138, Abbott A-121798, Abbott A 72694, Abbott A-80263, Biofor BF-397, Bristol-Myers Squibb BU-4601A, Carbazoycin C, Lagunamycin, Wellcome BW-70C, Ciba-Geigy CGS-26529, Warner-Lambert CI 1004, Warner-Lambert PD-136005, Warner-Lambert PD-145246, Eisai E 3040, Fujirebio F-1322, Fisons FPL-64170, Fujisawa FR 110302, Nippon Hypox HX 0386, Merck & Co L-699333, Merck Frosst L 739010, Lilly LY269415, Lilly LY 178002, Meiji Milk MM-7002, Hoechst Roussel P 8892, Hoechst Roussel P 8977, Hoechst Roussel HP977, SmithKline Beecham SB-202235, Green Cross SS-81-OH, Terumo Keio University TMK 685, American Home Products WAY-121520, American Home Products WAY-125007, Zeneca ZD 7717, Zeneca ZM 216800, Zeneca ZM 230487, 1,2-dihydro-n-(2-thiazolyl)-1-oxopyrrolo(3,2,1-kl)phenol-thiazin-1-carboxamide, Abbott A-65260, Abbott A-69412, Abbott-63162, American Home Products AHR-5333, Bayer Bay-q-1531, Boehringer Ingelheim BI-L-357, Boehringer Ingelheim BI-L-93BS, Boehringer Ingelheim BI-L 226XX, Bristol-Myers Squibb BMY-30094, Carbazomycin B, Wellcome BW 4C, Wellcome BW-B218C, Wellcome BW-B70C, Chauvin CBS-1114, Ciba-Geigy CGS-21595, Ciba-Geigy CGS-22745, Ciba-Geigy CGS-23885, Ciba-Geigy CGS 24891, Ciba-Geigy CGS-8515, Chiesi CHF-1909, Warner-Lambert CI-986, Warner-Lambert CI 987, Cirsiliol, Docebenon, DuPont Merck DuP-654, Eisai E 5110, Eisai E-6080, Green Cross EN-105, Enofelast, Epocarbazolin-A, Eprovafen, Evandamin, Forsythiasid, Fisons FPL 62064, Glaxo GR-80907, Zeneca ICI-211965, Isoflavane, Kyowa Hakko KF-8940, Merck & Co L-651392, Merck & Co L651896, Merck & Co L-652343, Merck & Co L-656224, Merck & Co L-670630, Merck & Co L-674636, Merck & Co L-691816, Lilly LY233569, Lilly LY-280810, Merck & Co MK-591, Merck & Co MK886, Nitrosoxacin-A, Ono ONO-5349, Ono ONO-LP-219, Ono ONOLP-269, Warner-Lambert PD-127443, Purdue Frederick PF-5901, Sandoz QA-208-199, Johnson & Johnson R-68151, Johnson & Johnson R-85355, Rhone-Poulenc Rorer Rev-5367, Revlon 5901, Rhone-Poulenc Rorer RG-5901-A, Rhone-Poulenc Rorer RG-6866, Roussel-Uclaf RU-46057, Searle SC-41661A, Searle SC-45662, Sandoz SDZ-210610, SmithKline Beecham SK&F-104351, SmithKline Beecham SK&F-104493, SmithKline Beecham SK&F-105809, Synthelabo SL-810433, Teijin TEI-8005, Terumo TMK-777, Terumo TMK-781, Terumo TMK-789, Terumo TMK-919, Terumo TMK-992, Teikoku Hormone TZI-2721, Teikoku Hormone TZI-41127, American Home Products WAY-120739, American Home Products WY 47288, American Home Products WY-48252, American Home Products WY-50295, Yoshitomi Y-19432, dihydroarachidonic acid, Merck MK571, Merck MK679, IC1207,968 and IC1204,219 (ICI), SC-41930, SC-51146, SC-37920, SC-53228, SC-50605 and SC-51146 (Searle), Wako AA-681, Wellcome BW755C, KCi 1404 [(4-methyl-2-pyridinyl)-1-piperazinyl)ethyl)-4H-pyrrolo(3,2,1-ij)quinolin-e, 15-HETE, Leflunomide (HWA486), 4-acylaminophenyl derivatives, Chamazulen (chamomile extract), poly-unsaturated fatty acids, VLM295 or LY293111 (Vanguard), 4,5-dihydro-1H-1,2,4-triazolquinone adducts of the type 1 a, b, c (N-adducts) and of the type 2 (C-adducts) as products of the reaction of quinones with N-alkyl- and N-arylhydrazones, terpenes with acetyl-11-keto-beta-boswellic acid (AKBA) as chemical lead and frankincense extract.

4. The method of claim 2, wherein the 5-lipoxygenase inhibitor is a non-steroidal inhibitor.

5. The method of claim 2, wherein the 5-lipoxygenase inhibitor is (±)-1-(1-benzo[b]thien-2-ylethyl)-1-hydroxyurea (Zileuton).

6. The method of claim 2, wherein the 5-lipoxygenase inhibitor is boswellic acid or a derivative thereof.

7. The method of claim 1, further comprising administering at least one anti-acne active ingredient in combination with the at least one lipoxygenase inhibitor.

8. The method of claim 7, wherein the anti-acne active ingredient is a retinoid.

9. The method of claim 1, wherein the administration is oral, topical oral and topical.

10. The method of claim 1, wherein the acne is inflammatory.

11. A method for treating acne, comprising administering to a mammal in need thereof an effective amount of a pharmaceutical composition comprising at least one lipoxygenase inhibitor and one pharmaceutically acceptable carrier.

12. The method of claim 11, wherein the lipoxygenase inhibitor is a 5-lipoxygenase inhibitor.
